(19) United States
(12) Patent Application Publication
(10) Pub. No.: US 2006/0160112 A1
(43) Pub. Date: Jul. 20, 2006

(54) Tissue inhibitor of metalloproteinase (TIMP) as a marker and predictor of cardiovascular disease and both cardiac and non-cardiac mortality

(76) Inventors: Erdal Cavusoglu, Douglaston, NY (US); Jonathan D. Marmur, New York, NY (US); David J. Pinsky, Ann Arbor, MI (US); Cyril Ruwende, Ann Arbor, MI (US)

Correspondence Address: DILWORTH & BARRESE, LLP, 333 EARLE OVINGTON BLVD., UNIONDALE, NY, 11553, US

(21) Appl. No.: 11/292,031

(22) Filed: Dec. 1, 2005

Related U.S. Application Data
(60) Provisional application No. 60/632,650, filed on Dec. 2, 2004.

(51) Int. Cl. C12Q 1/68; G01N 33/53; G01N 33/00

(52) U.S. Cl. 435/6; 435/7.1; 436/86

(57) ABSTRACT

The present invention is directed to a method for characterizing an individual's risk profile of developing vascular disease, for an adverse outcome in a patient, for mortality in an individual diagnosed with vascular disease by evaluating the level of tissue inhibitor of metalloproteinase-1 (TIMP-1) in a sample taken from the individual. The invention is also directed to a kit for characterizing the same.

### CROSS REFERENCES

This Application claims the benefit of U.S. Provisional Application No. 60/632,650 filed Dec. 2, 2004, the disclosure of which is incorporated herein by reference.

### BACKGROUND OF THE INVENTION

Abnormalities in the connective tissue component of the vessel wall have been implicated in the genesis of atherosclerosis and its associated complications.^{1,2} Connective tissue integrity depends on a balance between degradation and repair of the extracellular matrix. A family of enzymes known as the matrix metalloproteinases (MMPs) plays a major role in the degradation of collagen and other extracellular matrix macromolecules.² An important mechanism for the regulation of the activity of MMPs is via binding to a family of homologous proteins referred to as the tissue inhibitors of metalloproteinases (TIMPs).³ Under normal circumstances, the TIMPs are in delicate balance with the MMPs and matrix is digested in a highly regulated fashion. However, during certain disease states, including atherosclerosis, there is an imbalance between the activities of these two families of proteins leading to tissue destruction.^{4,5} Indeed, the MMPs and the TIMPs, through their effects on proteolysis and its inhibition, are now believed to play a critical role in extracellular remodeling during all phases of atherosclerosis, from its genesis and progression to the development of its acute complications.4, 6, 7 Furthermore, because these proteins are synthesized in response to cytokines,⁸ they may serve to link inflammation and atherosclerosis.

Therefore what is needed is a method for characterizing an individual's risk profile of developing vascular disease, for an adverse outcome in a patient, for mortality in an individual diagnosed with vascular disease by evaluating the level of tissue inhibitor of metalloproteinase-1 (TIMP-1) in a sample taken from the individual. What also is needed is a kit for characterizing each of the aforementioned risk profiles. The present invention provides these benefits.

### SUMMARY OF THE INVENTION

The present invention is directed to a method for characterizing an individual's risk profile of developing vascular disease by obtaining a biological sample from the individual and measuring the level of tissue inhibitor of metalloprotein (TIMP) in the biological sample. Once the level of tissue inhibitor of metalloprotein (TIMP) has been determined the obtained level of TIMP can be compared to a predetermined value or a control sample and the individual's risk profile of developing vascular disease is characterized based upon the TIMP level of the biological sample in comparison to the predetermined value or control sample.

The present invention is also directed to a method for characterizing the risk profile for an adverse outcome in a patient diagnosed with vascular disease by obtaining a biological sample from the patient and measuring the level of tissue inhibitor of metalloprotein (TIMP) in the sample. The level obtained is then compared with a predetermined value or a control sample and based on this comparison the patient's risk profile of having an adverse outcome based upon the TIMP level is determined.

Also provided by the present invention is a method for guiding therapy in a patient with a vascular disease comprising (a) obtaining a baseline level of tissue inhibitor of metalloprotein (TIMP) in a biological sample obtained from the patient, (b) administering to the patient a treatment for the vascular disease, (c) obtaining one or more subsequent biological samples from the patient, (d) measuring TIMP level in the one or more subsequent samples, (e) comparing the level of TIMP in the one or more subsequent samples with the baseline level; and (f) determining whether increased dosages or additional or alternative treatments are needed based on TIMP levels obtained in the one or more subsequent samples compared to the TIMP baseline level.

Another embodiment of the invention is directed to a kit for identifying an individual at risk for developing vascular disease, for predicting an adverse outcome in a patient diagnosed with vascular disease, for predicting all-cause mortality in an individual, or for guiding therapy in a patient with a vascular disease, the kit comprising an antibody which specifically binds a tissue inhibitor metalloprotein (TIMP), a listing of one or more predetermined values, and instructions for its use.

### BRIEF DESCRIPTION OF THE FIGURES

FIG. 1A is a chart depicting the Odds Ratio for all-cause mortality at 24 months.

FIG. 1B is a chart depicting the Odds Ratio for cardiac mortality at 24 months.

FIG. 1C is a chart depicting the Odds Ratio for MI at 24 months.

FIG. 2A is a graphical representation showing Survival Rate curves for All-Cause Mortality at 24 months by TIMP-1 intervals in the entire cohort.

FIG. 2B is a graphical representation showing Survival Rate curves for Cardiac Mortality at 24 months by TIMP-1 intervals in the entire cohort.

FIG. 3A is a graphical representation showing Survival Rate curves for All-Cause Mortality at 24 months by TIMP-1 intervals in ACS population.

FIG. 3B is a graphical representation showing Survival Rate curves for Cardiac Mortality at 24 months by TIMP-1 intervals in ACS population.

### DETAILED DESCRIPTION OF THE INVENTION

The present invention relates to the use of a family of proteins known as tissue inhibitors of metalloproteinases (TIMPs) in identifying individuals at risk for developing vascular disease, for predicting adverse outcomes, and for guiding therapy in such individuals. The present invention therefore provides new diagnostic tests that determine and utilize the levels of TIMP in an individual, patient or population. The tests are based upon the surprising discoveries described below.

In accordance with the present invention, it has been discovered that a single baseline measurement of TIMP is a powerful and independent predictor for the development of future myocardial infarction, and both cardiac and non-cardiac mortality. In addition, it has also been discovered that the TIMP-1/MMP-9 ratio is an independent predictor of cardiac death. The present invention, therefore provides methodologies for assessing the risk to individuals of developing vascular disease as well as for predicting adverse outcomes in those already diagnosed with the disease. These methodologies are useful in defining individuals who may benefit from further screening and testing for cardiovascular disease, and may be used to assess and guide therapy.

TIMP levels show more predictive power that other established biomarkers such as high-sensitivity C-reactive protein. The methods provided by the present invention are relatively easy to perform using small quantities of blood, serum, plasma, tissue or urine. TIMP levels may be assessed alone or in combination with MMP-9 or other recognized risk markers to establish a risk profile which guides diagnostic and therapeutic strategies. In this regard, the ratio of TIMP to MMP is likely important.

In a first embodiment of the invention, there is provided a method for characterizing an individual's risk profile of developing vascular disease. The method comprises obtaining a biological sample from the individual, measuring the level of tissue inhibitor of metalloprotein (TIMP) in the biological sample, comparing the level of TIMP to a predetermined value or a control sample, and characterizing the individual's risk profile of developing vascular disease based upon the TIMP level of the biological sample in comparison to the predetermined value or control sample. Preferably, the individual is apparently healthy. As used herein, the term “apparently healthy” means an individual who has not been previously diagnosed with vascular disease and/or who shows no symptoms of vascular disease.

As used herein, “predetermined value” can be a single value, multiple values, a single range or multiple ranges. For example, a predetermined value may be a single cut-off value such as a median or mean. A predetermined value may be established based upon comparative groups. An example of such an establishment is when the risk in a particular group of individuals is two times or double the risk in another defined group. A predetermined value may also be a range such as when a population is divided into groups such as a low-risk group, a medium-risk group and a high-risk group. Alternatively, a predetermined value may be based on a population grouped into quadrants, with the lowest quadrant composed of individuals with the lowest risk and the highest quadrant composed of individuals with the highest risk.

In accordance with the present invention, a predetermined value may be dependent upon the specific make-up of the selected population. Thus for example, sample populations may be divided based on various risk factors such as active tobacco use. An apparently healthy non-smoking group of individuals will have a different normal TIMP level range than a sample population of smokers (active tobacco use). Accordingly, a predetermined value for use in the present invention should relate to the type of sample population into which an individual falls. Appropriate ranges and groups may be created and selected using a routine level of skill in the art. Therefore, a predetermined value may be a plurality of predetermined TIMP level ranges and the comparing step comprises determining into which of the predetermined TIMP level ranges the individual falls.

In a method of characterizing an individual's risk profile for developing vascular disease, an elevated TIMP level in the biological sample compared to the predetermined value or control sample correlates with an increased likelihood that the individual will develop vascular disease. In this first embodiment of the invention, a predetermined value may be e.g., about 0 ng/ml. The predetermined value may also be about 3 ng/ml or higher, 5 ng/ml or higher or about 10 ng/ml.

Thus, for example, a TIMP level in a biological sample exhibiting a range of from about 0 to about 9 ng/ml likely indicates a low risk of developing vascular disease in that individual from which the biological sample was derived. A TIMP level in a biological sample exhibiting a range of from about 3 to about 5 ng/ml may indicate a slight risk of developing vascular disease in that individual from which the biological sample was derived. A TIMP level in a biological sample exhibiting a range of from about 5 to about 10 ng/ml may indicate an increased risk of developing vascular disease in that individual from which the biological sample was derived. A TIMP level in a biological sample exhibiting greater than 10 ng/ml indicates an even greater risk of developing vascular disease in the individual from which the biological sample was derived.

In another aspect of the invention, there is provided a method for characterizing the risk profile for an adverse outcome in a patient diagnosed with vascular disease. The method comprises the steps of: obtaining a biological sample from the patient, measuring the level of tissue inhibitor of metalloprotein (TIMP) in the sample, comparing the level of TIMP to a predetermined value or a control sample; and characterizing the patient's risk profile of having an adverse outcome based upon the TIMP level of the biological sample in comparison to the predetermined value or control sample. As described above, a predetermined value may be plurality of predetermined TIMP level ranges and the comparing step comprises determining into which of the predetermined TIMP level ranges the individual falls.

For example, a patient exhibiting elevated TIMP levels compared to the predetermined value or control sample indicates an increased likelihood for that patient having an adverse outcome. Such a finding would indicate the need for more drastic or extreme medical intervention and/or closer monitoring of the patient in order to avoid the adverse outcome. Examples of adverse outcome include but are not limited to stroke, myocardial infarction, cardiac death, and non-cardiac death.

As described above, a predetermined value can be a single value, multiple values, a single range or multiple ranges. In this embodiment of the invention, a predetermined value may be about 10 ng/ml or higher, 30 ng/ml or higher, or about 60 ng/ml or higher.

In still another aspect of the invention, there is provided a method for characterizing the risk profile for mortality in an individual. The method comprises the steps of: obtaining a biological sample from the individual, measuring the level of tissue inhibitor of metalloprotein (TIMP) in the biological sample, comparing the level of TIMP to a predetermined value or a control sample; and characterizing the individual's risk profile for mortality based upon the TIMP level of the biological sample in comparison to the predetermined value or control sample. The biological sample can either be a sample taken specifically to measure the level of tissue inhibitor of metalloprotein (TIMP) in the sample or can be a sample taken as part of a risk factor profile panel. In other words, when a serum/cell sample is taken from a patient to check other risk factors such as cholesterol, C-reactive proteins, glucose as well as others, the assay of the present invention may also be performed on this sample as part of the risk factor work up.

An increased level of TIMP in the biological sample when compared to the predetermined value or control sample, correlates with an increased likelihood of mortality in the individual. In the alternative, measuring TIMP activity in addition to or instead of TIMP levels may also be used to determine the likelihood of mortality in an individual.

Useful predetermined values for practicing this aspect of the invention include but are not limited to about 50 ng/ml, 80 ng/ml or lower, or about 100 ng/ml or lower. Thus, an individual having a TIMP level greater than about 80-100 ng/ml is strongly predictive of all cause and cardiac mortality.

The present invention still further provides a method for guiding therapy in a patient with a vascular disease. By “guiding therapy” is meant monitoring the patient with respect to his or her TIMP levels and basing the patient's treatments or therapies on the results of such monitoring. The method comprises the steps of: obtaining a baseline level of tissue inhibitor of metalloprotein (TIMP) in a biological sample obtained from the patient, administering to the patient a treatment for the vascular disease; obtaining one or more subsequent biological samples from the patient; measuring TIMP level in the one or more subsequent samples, comparing the level of TIMP in the one or more subsequent samples with the baseline level, and determining whether increased dosages or additional or alternative treatments are needed based on TIMP levels obtained in the one or more subsequent samples compared to the TIMP baseline level.

Thus for example, a patient exhibiting an absence of a detectable decrease or an insignificant decrease in TIMP levels after treatment would be a candidate for increased dosage of the treatment, or for additional or alternative treatments.

By “treatments” is meant the administration of a particular drug or therapy with the aim of reducing TIMP levels and/or other symptoms of vascular disease. Examples of treatments include but are not in any way limited to anti-coagulant therapy, administration of a lipid lowering drug, anti-platelet therapy, anti-thrombosis therapy, administration of an anti-inflammatory drug, or arterial revascularization. Examples of arterial revascularization include surgery or angioplasty.

As used herein, “biological sample” means any sample taken from an animal subject, including but not limited to a blood sample, serum sample, plasma sample, tissue sample, cell sample, or urine sample. Tissue samples may include material from biopsies. Cell samples may include one or more single or isolated cells, and cell fragments. Preferably, the animal is human.

Also as used herein, “vascular disease” may include a number of well recognized infirmities, including but not limited to artherosclerosis, congestive heart failure, myocardial infarction, chronic renal insufficiency, valvular heart disease or coronary artery disease.

In accordance with the present invention, TIMP may be detected in biological samples using methods well known in the art. As described hereinabove, TIMP levels may be determined by contacting a biological sample from the patient or individual with an antibody that specifically binds a tissue inhibitor metalloprotein (TIMP), allowing the formation of a complex between the antibody and the TIMP in the sample, detecting the presence and measuring the level of antibody-TMP complex in the sample. In a preferred embodiment, the antibody carries a detectable label, and the detection step is performed by detecting the presence of the label after separation of the antibody-TIMP complex from the uncomplexed antibody.

TIMP may also be detected and levels measured using other well known methods such as by flow cytometry, or HPLC. TIMP mRNA levels which correlate to TIMP protein levels may be detected by hybridization to an appropriate probe or by using PCR technology. The amino acid sequence for TIMPS are known (3) and probes or primers may be designed based on the known amino acid sequences using the universal code and employing well known methodologies. Of the available TMP isoforms (TIMP1 to TIMP4), TIMP-1 is preferably used in the present invention.

In accordance with the present invention, the measuring of TIMP does not have to be quantitative; as long as the level of TIMP may be compared to a predetermined value, TIMP levels are effectively measured. Thus, color charts corresponding to predetermined TIMP levels when colored or fluorescent reagents are used, as well as calorimetric or spectrophotometric values are useful for practicing the present invention.

In addition to the methods provided herein, there are provided kits which are useful in performing such methods. Thus, there are provided kits for identifying an individual at risk for developing vascular disease, for predicting an adverse outcome in a patient diagnosed with vascular disease, for predicting all-cause mortality in an individual, or for guiding therapy in a patient with a vascular disease. In one embodiment, the kit comprises an antibody which specifically binds a tissue inhibitor metalloprotein (TIMP), a listing of one or more predetermined values, and instructions for its use. In an alternative embodiment, the kit comprises one or more reagents for detecting cell-bound TIMP by flow cytometry, a listing of one or more predetermined values, and instructions for its use. In still another embodiment, the kit comprises one or more probes or primers for detecting TIMP mRNA in a cell sample, a listing of one or more predetermined values, and instructions for its use. In still another embodiment, the kit comprises one or more reagents for detecting TIMP in a biological sample by HPLC, a listing of one or more predetermined values, and instructions for its use. If desired, the kit may comprise any combination of the above-described reagents, probes, or primers so that the user of the kit may use alternative methods to detect and measure TIMP levels.

Preferably, the kit measures the TIMP-1 isoform. In a further embodiment, the kit may comprise one or more containers for collecting and/or holding a blood, serum, plasma, urine or tissue sample.

By “listing of one or more predetermined values” means any listing, chart, table, color chart, or the like which indicates the relevant predetermined value for the particular method using the kit. There may be other materials contained in the kit such as a control samples for completing the comparison step. The control sample will have a predetermined TIMP value and can be used as a standard against which TIMP levels in the biological samples are compared. Such comparison may be made either directly by eye, or by instrumentation such as HPLC, colorimeter, or spectrophotometer.

In order to illustrate various illustrative embodiments of the present inventions, the following examples are provided.

### EXAMPLES

To study the role of these molecules in atherosclerosis, we examined their levels in a well-characterized and prospectively-followed heterogeneous population of patients undergoing coronary angiography for a variety of indications. The relationship between these markers and traditional coronary artery disease (CAD) risk factors, other recognized prognostic biomarkers, the presenting coronary syndrome, angiographic severity of CAD, left ventricular (LV) systolic function, as well as overall prognosis. Of this large class of metalloproteinases and their inhibitors, we chose to study MMP-9, which is associated with plaque instability,⁵ and TIMP-1, which is known to inhibit most active MMPs, to be expressed in atherosclerotic plaques,^{9, 10} and for which there is strong experimental evidence of a direct pro-atherosclerotic role.¹¹

### METHODS

Study Design

Between January 1999 and October 2002, 389 male patients undergoing diagnostic coronary angiography for a variety of indications were enrolled in the study. The patients with chest pains were risk stratified into those with and without an acute coronary syndrome to determine if TIMP-1 was still an independent predictor of all-cause mortality in both subgroups. The only exclusion criteria for participation in the study were active gastrointestinal bleeding or the presence of anemia, which was defined as a hemoglobin concentration less than 8 gm/dL. Clinical and demographic information was obtained by interview as well as review of computerized medical records. Fasting blood was obtained from all patients at the time of angiography for subsequent analysis. The patients were then followed prospectively for the next 24 months for the development of clinical events.

TIMP-1 and matrix metalloproteinase-9 (MMP-9) levels, along with a number of other biomarkers, were measured in 389 male patients undergoing coronary angiography for a variety of indications in the Veterans Administration Medical Center. Patients were followed for the occurrence of all-cause mortality, cardiac mortality, and myocardial infarction (MI) at 24 months.

Blood Sampling

After an overnight fast of at least 12 hours, blood was obtained from all patients enrolled in the study. Blood was collected from the arterial sheath (after a 5 mL discard) at the time of angiography but prior to the injection of contrast material. Blood was immediately placed into vacutainer tubes, spun at 10 g for 20 minutes in a cold centrifuge and the plasma aliquoted into multiple 1.5 mL eppendorfs. The samples were subsequently stored at −70° C. until analysis at a later date.

Laboratory Methods

Aliquoted plasma samples stored at −70° C. were thawed and, using commercially available ELISA kits, the levels of the following biomarkers were measured: TIMP-1 (Oncogene Research Products), MMP-9 (Oncogene Research Products), high-sensitivity C-reactive protein (hs-CRP; Life Diagnostics), myeloperoxidase (MPO; Assay Designs), and plasminogen activator inhibitor-1 (PAI-1; Diapharma). The sensitivities of the TIMP-1, MMP-9, hs-CRP, MPO and PAI-1 assays were 0.0096 ng/mL, 0.1 ng/mL, 0.1 mg/L, 0.13 ng/mL, and 0.5 ng/mL, respectively.

Definition of Risk Factors and Clinical Syndromes

Diabetes mellitus was defined as clinically known and treated diabetes mellitus. Patients were diagnosed as hypertensive if they were documented to have a blood pressure greater than 140/90 mmHg on more than 2 occasions or were already on anti-hypertensive therapy. Hyperlipidemia was diagnosed in patients who had been given lipid-lowering medication or had a history of total cholesterol levels>240 mg/dL.¹² Smoking was defined as the inhaled use of cigarettes, cigars or pipes in any quantity. Smokers were classified as former only if they had not smoked at all in the 6 months preceding the date of angiography. Obesity was defined as a body mass index (BMI)>30 kg/m². Chronic renal insufficiency (CRI) was defined as a serum creatinine≧2.0 mg/dL. Congestive heart failure (CHF) on presentation was defined as the presence of either radiographic or clinical evidence of pulmonary venous congestion within the preceding 24 hours of angiography. Myocardial infarction (MI) on presentation was diagnosed by a history of chest discomfort and either troponin I>1.0 ng/mL or troponin T>0.10 ng/mL.

Angiographic Scoring System

Using visual estimation of stenosis severity, angiograms were scored as follows: any obstructive lesion≧50% in one of the three coronary arteries or their major (≧2.5 mm) branches received a score of 1. Multiple obstructive lesions≧50% in a single artery or one of its major branches still only received a score of 1, except in the case of a left dominant system. In the case of a left dominant system, an obstruction≧50% in the proximal portion of the left circumflex artery received a score of 2. Similarly, in a left dominant system, an obstruction≧50% in both a large obtuse marginal branch and a left posterior descending artery (PDA) also received a score of 2. On the other hand, an obstruction≧50% in a left dominant system which was isolated to either a left PDA or an obtuse marginal (but not both) received a score of 1. An obstruction of≧50% in the left main coronary artery received a score of 2 in a right dominant system and 3 in a left dominant system. A bypass graft with an obstructive lesion≧50% received a score of 1. Patent grafts or those with an obstructive lesion≦50% received a score of 0.

Angiograms were scored by 2 different angiographers working independently. Any differences in interpretation (7%) were subsequently reconciled. The operators scoring the angiograms were blinded to the results of any subsequent laboratory analysis or to the development of clinical events at long-term follow-up.

LV systolic function was assessed by contrast ventriculography and categorized as normal (ejection fraction [EF]≧55%), mildly-(EF 45-54%), moderately-(EF 31-44%), or severely-reduced (EF≦30%).

Clinical Endpoints

Patients were followed for the occurrence of death (all-cause and cardiac) and MI. MI during follow-up (i.e., as a clinical outcome) was defined by a history of chest pain with an associated elevation of either troponin I>1.0 ng/mL or troponin T>0.1 ng/mL. In the case of a fatal MI where enzymatic confirmation was not possible, the diagnosis was made on the basis of either a death certificate or a hospital record documenting MI as the cause of death. Follow-up was obtained via a combination of telephone contact, review of the computerized medical records, and subsequent clinic visits and hospitalizations. A death was classified as cardiac if the predominant and immediate cause was related to myocardial infarction or ischemia, arrhythmia, refractory congestive heart failure, or if the death was sudden and unexpected in nature. A death was categorized as non-cardiac if the major underlying pathophysiologic process leading to the demise was not related to the cardiovascular system, such as metastatic malignancy, sepsis, liver failure, or pulmonary embolism. The information regarding the etiology and date of death was obtained using the following modalities: review of the death certificate, screening of the social security death index, conversation with the next of kin and/or primary physician, and, most commonly, review of the national VA computerized medical records. In each and every case, the cause and date of death were confirmed with the use of more than one modality.

Statistical Analysis

The study population was divided into 3 groups based on the 25^{th} and 75^{th} percentiles of TIMP-1 values as follows: (i) lower quartile (TIMP-1≦66.5 ng/mL), (ii) interquartile (TIMP-1>66.5 ng/mL and≦100 ng/mL), and (iii) upper quartile group (TIMP-1>100 ng/mL).

Summary statistics for continuous variables were recorded as mean and standard deviation and the comparison between the three groups was performed with the non-parametric Kruskall-Wallis test. Categorical data were summarized as frequencies and percentages, and the comparisons between the 3 groups were performed with Pearson chi-square test or Fisher's exact test. The association between biomarkers and angiographic score was assessed with Spearman rank correlation.

Time-to-event at 24 months was analyzed with Kaplan-Meier curves for the individual endpoints of all-cause mortality, cardiac mortality, and MI. Comparison between the 3 groups was performed with the Log rank test.

The predictors of all-cause mortality, cardiac mortality, and MI at 24 months were identified with univariate logistic regression and the results presented with odds ratios and 95% confidence intervals. Multiple logistic regressions with backward selection of variables and calculation of odds ratios with the respective 95% confidence intervals were used to identify the independent predictors.

All analyses used two-sided tests with overall significance level, alpha=0.05.

### RESULTS

Base-Line Characteristics

A total of 389 patients were enrolled in the study with a mean follow up of 41±18 months (range from 2 days to 68 months; median 45 months). Two-year follow up data were available for 97% of the patients. The baseline clinical, laboratory and angiographic characteristics of the study population stratified by lower, inter-, and upper quartile TIMP-1 values are shown in Table 1.

Association of TIMP-1 with Baseline Clinical Variables and Other Biomarkers

Elevated TIMP-1 levels were seen in association with older age, active tobacco use (borderline significance), CHF on presentation, MI on presentation, and CRI (Table 1). In addition, TIMP-1 levels were also positively correlated with the erythrocyte sedimentation rate (ESR), as well as with the levels of MMP-9, hs-CRP, PAI-1, and MPO. In contrast, family history and hyperlipidemia were inversely correlated with TIMP-1 values. There was no association with diabetes mellitus, hypertension, obesity, race, or troponin levels.

Association of TIMP-1 with Angiographic Score

By multivariate analysis, age (p<0.001), hyperlipidemia (p<0.001), chronic renal insufficiency (p=0.006), LV function (p=0.01), and the ratio of TIMP-1 to MMP-9 (p=0.02) were predictive of the extent of angiographic CAD. Although weak, TIMP-1 levels correlated with CAD score (r=0.16; p<0.001). With respect to LV systolic function, there was a greater proportion of patients with LV dysfunction in the highest TIMP-1 quartile (Table 1).

Association of TIMP-1 with Clinical Outcomes at 24 Months: TIMP-1 as a Predictor of All-Cause Mortality, Cardiac Mortality, and Myocardial Infarction.

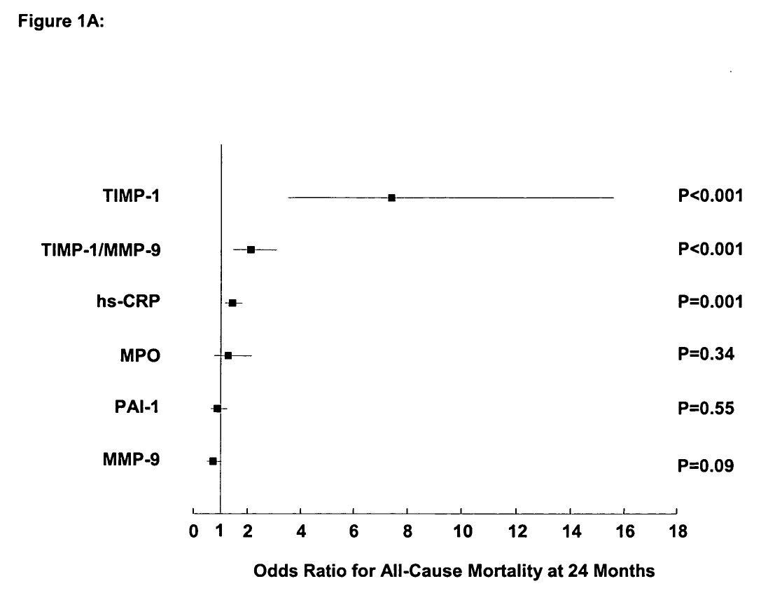

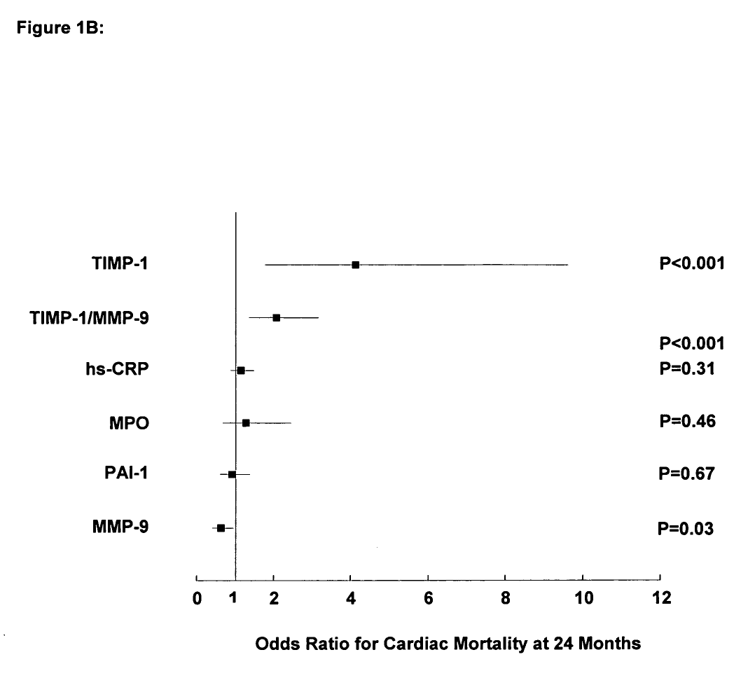

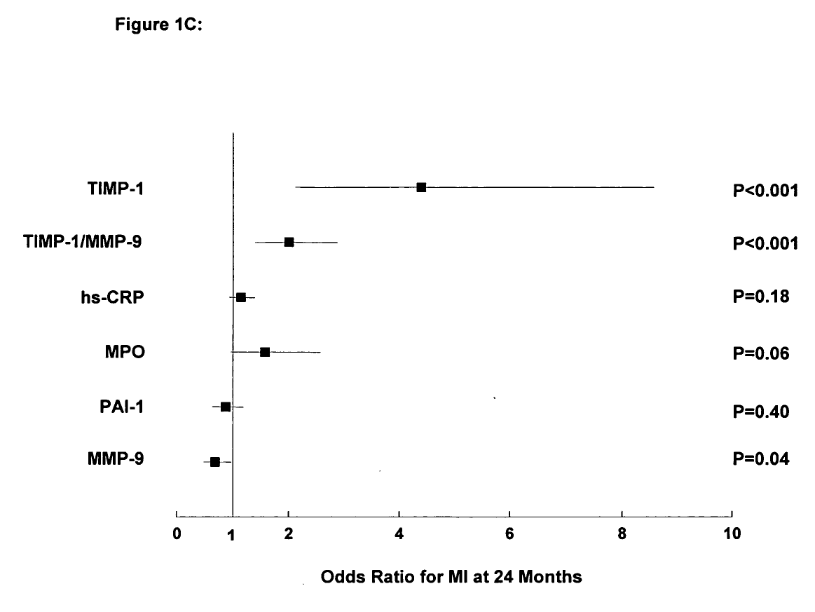

For the 97% of patients in whom 24 month follow-up data were available, there was a total of 51 deaths (13%), of which 31 (61%) were classified as cardiac in etiology. Similarly, 61 (16%) had developed an MI (fatal or non-fatal) by 24 months. The results of univariate and multivariate analyses for the prediction of all-cause mortality are presented in Tables 2 and 3, respectively. Using the same variables listed in Table 2, univariate predictors for the outcomes of cardiac mortality and MI at 24 months were identified and then entered into separate multivariate models, the results of which are shown in Tables 4 and 5, respectively. Based on univariate analysis, the unadjusted relative risk for the development of each of the 3 major endpoints of all-cause mortality, cardiac mortality and MI was greater for TIMP-1 than for any other biomarker (FIG. 1). Furthermore, TIMP-1 or its ratio with MMP-9 (TIMP-1/MMP-9), was the only biomarker found to be an independent predictor of each of these individual endpoints on multivariate analysis. When the analysis for MI was restricted to non-fatal events (n=51), TIMP-1 was still the only independently predictive biomarker [OR 1.10; 95% CI, 1.04 to 1.17; p<0.001].

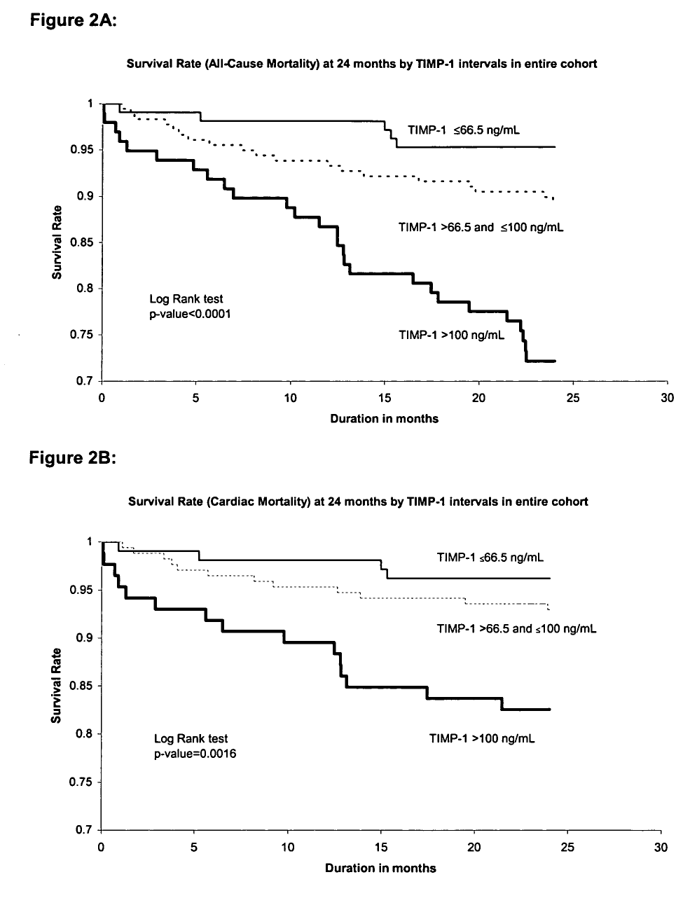

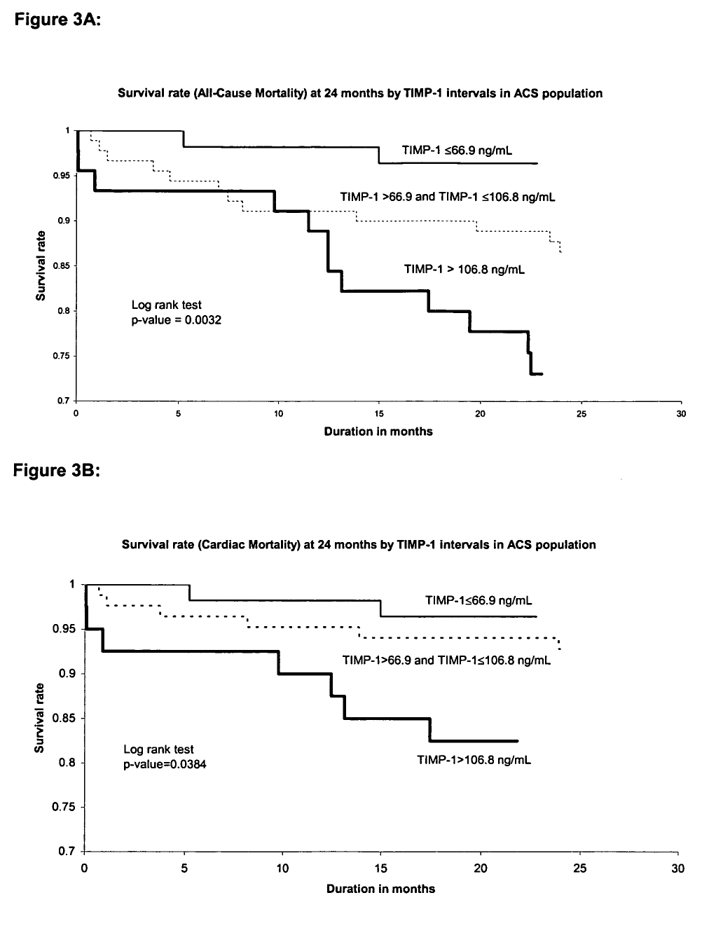

Using the 25^{th} and 75^{th} percentiles as prespecified cut-off points for TIMP-1 levels, Kaplan-Meier curves were derived for patients with TIMP-1 values <66.5 ng/mL, between 66.5 and 100 ng/mL, and>100 ng/mL (FIG. 2). Kaplan-Meier plots demonstrated a significant increase in mortality with increasing TIMP-1 values. In particular, TIMP-1 values exceeding 100 mg/dl were strongly predictive of all-cause and cardiac mortality. For all-cause mortality, the death rates at 24 months in the three groups were 95.3%, 89.3% and 72.2%, respectively (p<0.001). For cardiac death, the rates were 96.2%, 93.0% and 82.6%, respectively (p=0.002), while for non-cardiac death they were 99.0%, 96.4% and 87.4%, respectively (p<0.001).

### DISCUSSION

The correlation between TIMP-1 levels and angiographic extent of CAD, as well as the elevation of TIMP-1 levels in patients presenting with acute MI, support a role for TIMP-1 in the initiation/progression and clinical manifestations of atherosclerosis, respectively. MMP-mediated degradation of endothelial cell basement membrane, and its regulation by the TIMP system, has been implicated in the genesis of the early atherosclerotic lesion via a decreased endothelial barrier function to inflammatory cells during diapedesis.^{2, 6, 7, 13} There is also evidence that TIMP-1 itself may directly promote the development of atherosclerotic lesions.¹¹ In addition, an emerging concept is that variations in TIMP/MMP expression and the resultant net proteolytic activity affect the type and extent of arterial remodeling,^{14, 15} which is now believed to be a major determinant in the progression of atherosclerosis.^{16, 17} The observation that TIMP-1 levels are elevated in patients with MI are in accord with the findings of a smaller Japanese study¹⁸ and are consistent with the growing evidence implicating macrophages and matrix degradation in the etiology of plaque rupture.^{4, 5, 19, 20} Studies have reported increased expression of MMP-1, MMP-3, MMP-9 and TIMP-1 in the shoulder region of atherosclerotic plaques where they co-localize with infiltrating macrophages.^{9, 21} In the atherosclerotic plaque, TIMP-1 may represent both a marker of destabilizing inflammatory activity as well as a response to it, akin to the leukocytosis observed in response to infections.

The most significant finding of the study is that TIMP-1 levels were powerful predictors of the most important expression of the atherosclerotic disease process, namely the clinical outcomes of death and myocardial infarction. Of the biomarkers assayed in our study (i.e., hs-CRP, MPO, TIMP-1, MMP-9 and PAI-1), only TIMP-1 was predictive of the development of myocardial infarction at 24 months on multivariate analysis. Similarly, TIMP-1 was the only biomarker predictive of all-cause and cardiac mortality on multivariate analysis.

The ability to predict mortality using a single baseline determination of TIMP-1 in a heterogeneous population of patients referred for cardiac catheterization, including a significant non-acute coronary syndrome portion, is notable, and may relate to the elevated risk of the study population.

The elevated baseline cardiovascular risk profile of our study population is manifest in the clinical, angiographic, and laboratory data. For example, the mean hs-CRP value for this cohort (23.8 mg/L) is greater than a previously reported level (10 mg/L) used to define a population at increased risk.²⁸ Using a multivariate model that included other inflammatory and thrombotic biomarkers, TIMP-1 was statistically the most powerful predictor of death and MI (as individual endpoints). These data raise the possibility that TIMP-1 is proximately, or perhaps even causally, related to the underlying atherosclerotic pathophysiology. For example, TIMP-1 is known to induce proliferation in a variety of cell types by mechanisms independent of matrix metalloproteinase inhibition.²⁹ Recently, TIMP-1 has also been demonstrated to have a mitogenic effect on human aortic smooth muscle cells, suggesting that it may directly contribute to the excessive smooth muscle cell proliferation seen in association with atherosclerosis.³⁰ Finally, TIMP-1 is known to promote angiogenesis,³¹⁻³³ regulate apoptosis,³⁴ and to amplify inflammation.³⁵ Thus, in addition to being a potential marker of inflammation, TIMP-1 may also be directly involved in the pathogenesis of atherosclerosis and its complications.

It was also found that TIMP-1 retained its predictive value in patients with varying risk profiles. In other words, TIMP-1 remained an independent and powerful predictor of all-cause mortality in both the ACS (n=193) and non-ACS (n=196) subgroups (despite a nearly 50% reduction in sample size for each subgroup compared to the original cohort). This conclusion was reached by a separate analyses performed for both the ACS and non-ACS chest pain groups using the same univariate and multivariate models used to predict outcome for the entire cohort.

Furthermore, in the ACS population, the ratio of TIMP-1 with MMP-9 continued to be an independent predictor (by multivariate analysis) of each of the individual endpoints of cardiac mortality and myocardial infarction. Thus, despite the fact that the ACS group had higher baseline TIMP-1 values compared to the non-ACS group (median of 81.1 vs. 78.3, respectively; p<0.05) and was clearly a higher risk cohort, the independent predictive power of TIMP-1 (by multivariate analysis) was still preserved in this subpopulation. The ACS group was indeed a higher risk group, compared to the non-ACS group, as manifest by a higher incidence of all-cause mortality (13.6% vs. 6.5%, p<0.05 ), any MI (19.8% vs. 9.3%, p<0.05), and non-fatal MI (18.2% vs. 7.3%, p<0.05).

In addition, the ACS group had higher baseline hs-CRP values compared to the non-ACS chest pain group (median of 13.2 vs. 6.8, respectively; p<0.0001), further supporting the claim that these were two subpopulations with disparate risk profiles. Finally, as was the case for the entire cohort of patients, it is important to point out that TIMP-1 was better at risk prognostication than either hs-CRP or myeloperoxidase in each of these two subgroups (ACS and non-ACS). In other words, the higher risk ACS group, TIMP-1 remained an independent predictor of all-cause mortality while its ratio with MMP-9 was independently predictive of each of the individual endpoints of cardiac mortality and MI. Even in the lower risk non-ACS population of chest pain patients, TIMP-1 was still an independent predictor of all-cause mortality at 24 months. These findings attest to the powerful prognostic power of TIMP-1 in patients with chest pain associated with varying risk profiles.

### CONCLUSION

In conclusion, TIMP-1 appears to be a particularly important inflammatory marker/mediator by virtue of its correlation with the entire spectrum of atherosclerosis and, most importantly, by its predictive power for the hard endpoints of death and MI. In particular, TIMP-1 was found to be an independent predictor of all-cause mortality in patients having chest pain regardless of whether the patients had or did not have an acute coronary syndrome.

### REFERENCES

1. Loftus I M, Naylor A R, Bell P R, Thompson M M. Matrix metalloproteinases and atherosclerotic plaque instability. Br J Surg 2002; 89:680-94.
2. Galis Z S, Khatri J J. Matrix metalloproteinases in vascular remodeling and atherogenesis: the good, the bad, and the ugly. Circ Res 2002; 90:251-62.
3. Brew K, Dinakarpandian D, Nagase H. Tissue inhibitors of metalloproteinases: evolution, structure and function. Biochim Biophys Acta 2000; 1477:267-83.
4. Shah P K, Falk E, Badimon J J, et al. Human monocyte-derived macrophages induce collagen breakdown in fibrous caps of atherosclerotic plaques. Potential role of matrix-degrading metalloproteinases and implications for plaque rupture. Circulation 1995; 92:1565-9.
5. Brown D L, Hibbs M S, Kearney M, Loushin C, Isner J M. Identification of 92-kD gelatinase in human coronary atherosclerotic lesions. Association of active enzyme synthesis with unstable angina. Circulation 1995; 91:2125-31.
6. Amorino G P, Hoover R L. Interactions of monocytic cells with human endothelial cells stimulate monocytic metalloproteinase production. Am J Pathol 1998; 152:199-207.
7. Romanic A M, Madri J A. The induction of 72-kD gelatinase in T cells upon adhesion to endothelial cells is VCAM-1 dependent. J Cell Biol 1994; 125:1165-78.
8. Galis Z S, Muszynski M, Sukhova G K, et al. Cytokine-stimulated human vascular smooth muscle cells synthesize a complement of enzymes required for extracellular matrix digestion. Circ Res 1994; 75:181-9.
9. Galis Z S, Sukhova G K, Lark M W, Libby P. Increased expression of matrix metalloproteinases and matrix degrading activity in vulnerable regions of human atherosclerotic plaques. J Clin Invest 1994; 94:2493-503.
10. Orbe J, Fernandez L, Rodriguez J A, et al. Different expression of MMPs/TIMP-1 in human atherosclerotic lesions. Relation to plaque features and vascular bed. Atherosclerosis 2003; 170:269-76.
11. Silence J, Collen D, Lijnen H R. Reduced atherosclerotic plaque but enhanced aneurysm formation in mice with inactivation of the tissue inhibitor of metalloproteinase-1 (TIMP-1) gene. Circ Res 2002; 90:897-903.
12. Blankenberg S, Rupprecht H J, Bickel C, et al. Cytomegalovirus infection with interleukin-6 response predicts cardiac mortality in patients with coronary artery disease. Circulation 2001; 103:2915-21.
13. Rosenberg G A, Estrada E Y, Dencoff J E. Matrix metalloproteinases and TIMPs are associated with blood-brain barrier opening after reperfusion in rat brain. Stroke 1998; 29:2189-95.
14. Mason D P, Kenagy R D, Hasenstab D, et al. Matrix metalloproteinase-9 overexpression enhances vascular smooth muscle cell migration and alters remodeling in the injured rat carotid artery. Circ Res 1999; 85:1179-85.
15. Pasterkamp G, Schoneveld A H, Hijnen D J, et al. Atherosclerotic arterial remodeling and the localization of macrophages and matrix metalloproteases 1, 2 and 9 in the human coronary artery. Atherosclerosis 2000; 150:245-53.
16. Komowski R, Mintz G S, Lansky A J, et al. Paradoxic decreases in atherosclerotic plaque mass in insulin-treated diabetic patients. Am J Cardiol 1998; 81:1298-304.
17. Mintz G S, Kent K M, Pichard A D, Satler L F, Popma J J, Leon M B. Contribution of inadequate arterial remodeling to the development of focal coronary artery stenoses. An intravascular ultrasound study. Circulation 1997; 95:1791-8.
18. Inokubo Y, Hanada H, Ishizaka H, Fukushi T, Kamada T, Okumura K. Plasma levels of matrix metalloproteinase-9 and tissue inhibitor of metalloproteinase-1 are increased in the coronary circulation in patients with acute coronary syndrome. Am Heart J 2001; 141:211-7.
19. Kai H, Ikeda H, Yasukawa H, et al. Peripheral blood levels of matrix metalloproteases-2 and -9 are elevated in patients with acute coronary syndromes. J Am Coll Cardiol 1998; 32:368-72.
20. Inoue T, Kato T, Takayanagi K, et al. Circulating matrix metalloproteinase-1 and -3 in patients with an acute coronary syndrome. Am J Cardiol 2003; 92:1461-4.
21. Sukhova G K, Schonbeck U, Rabkin E, et al. Evidence for increased collagenolysis by interstitial collagenases-1 and -3 in vulnerable human atheromatous plaques. Circulation 1999; 99:2503-9.
22. Brennan M L, Penn M S, Van Lente F, et al. Prognostic value of myeloperoxidase in patients with chest pain. N Engl J Med 2003; 349:1595-604.
23. de Lemos J A, Morrow D A, Sabatine M S, et al. Association between plasma levels of monocyte chemoattractant protein-I and long-term clinical outcomes in patients with acute coronary syndromes. Circulation 2003; 107:690-5.
24. Ridker P M, Buring J E, Rifai N. Soluble P-selectin and the risk of future cardiovascular events. Circulation 2001; 103:491-5.
25. Blankenberg S, Tiret L, Bickel C, et al. Interleukin-18 is a strong predictor of cardiovascular death in stable and unstable angina. Circulation 2002; 106:24-30.
26. Blankenberg S, Rupprecht H J, Bickel C, et al. Circulating cell adhesion molecules and death in patients with coronary artery disease. Circulation 2001; 104:1336-42.
27. Lindahl B, Toss H, Siegbahn A, Venge P, Wallentin L. Markers of myocardial damage and inflammation in relation to long-term mortality in unstable coronary artery disease. FRISC Study Group. Fragmin during Instability in Coronary Artery Disease. N Engl J Med 2000; 343:1139-47.
28. Ridker P M, Cook N. Clinical usefulness of very high and very low levels of C-reactive protein across the full range of Framingham Risk Scores. Circulation 2004; 109:1955-9.
29. Chesler L, Golde D W, Bersch N, Johnson M D. Metalloproteinase inhibition and erythroid potentiation are independent activities of tissue inhibitor of metalloproteinases-1. Blood 1995; 86:4506-15.
30. Akahane T, Akahane M, Shah A, Thorgeirsson U P. TIMP-1 stimulates proliferation of human aortic smooth muscle cells and Ras effector pathways. Biochem Biophys Res Commun 2004; 324:440-5.
31. Lafleur M A, Handsley M M, Edwards D R. Metalloproteinases and their inhibitors in angiogenesis. Expert Rev Mol Med 2003; 5:1-39.
32. Thorgeirsson U P, Yoshiji H, Sinha C C, Gomez D E. Breast cancer; tumor neovasculature and the effect of tissue inhibitor of metalloproteinases-1 (TIMP-1) on angiogenesis. In Vivo 1996; 10:137-44.
33. Yamada E, Tobe T, Yamada H, et al. TIMP-1 promotes VEGF-induced neovascularization in the retina. Histol Histopathol 2001; 16:87-97.
34. Guedez L, Mansoor A, Birkedal-Hansen B, et al. Tissue inhibitor of metalloproteinases 1 regulation of interleukin-10 in B-cell differentiation and lymphomagenesis. Blood 2001; 97:1796-802.
35. Apparailly F, Noel D, Millet V, et al. Paradoxical effects of tissue inhibitor of metalloproteinases 1 gene transfer in collagen-induced arthritis. Arthritis Rheum 2001; 44:1444-54.

Data are presented as frequencies and percentages for categorical variables, and as means (with standard deviations) and medians (with interquartile ranges) for continuous variables. Asterisk (*) denotes variables (including TIMP-1) with a skewed distribution.

Abbreviations: ESR=erythrocyte sedimentation rate; hs-CRP=high-sensitivity C-reactive protein; MPO=myeloperoxidase; PAI-1=plasminogen activator inhibitor-1; MMP-9=matrix metalloproteinase-9; TIMP-1=tissue inhibitor of metalloproteinase-1.

TABLE I
Baseline characteristics

| | | | | | | |
|---|---|---|---|---|---|---|
| | | | Interquartile | | | |
| | | Lower quartile | [TIMP > 66.5 | Upper quartile | Total | |
| | | [TIMP ≦ 66.5] | and ≦100] | [TIMP > 100] | population | p-value |
| Characteristics | Statistics | N = 108 | N = 183 | N = 98 | N = 389 | for trend |

| | | | | | | |
|---|---|---|---|---|---|---|
| Age | Mean (std) | 63.7 (9.6) | 65.2 (10.0) | 67.4 (10.0) | 65.3 (10.0) | 0.02 |
| Race | Black N (%) | 36 (33.3) | 56 (30.6) | 30 (30.6) | 122 (31.4) | 0.99 |
| | Hispanic | 30 (27.8) | 52 (28.4) | 29 (29.6) | 111 (28.5) | |
| | White | 42 (38.9) | 75 (41.0) | 39 (39.8) | 156 (40.1) | |
| Family history | N (%) | 32 (29.6) | 49 (26.8) | 15 (15.3) | 96 (24.7) | 0.04 |
| of CAD | | | | | | |
| Diabetes | N (%) | 47 (43.5) | 76 (41.5) | 46 (46.9) | 169 (43.4) | 0.68 |
| Hypertension | N (%) | 85 (78.7) | 155 (84.7) | 85 (86.7) | 325 (83.6) | 0.25 |
| History of | N (%) | 86 (79.6) | 146 (79.8) | 84 (85.7) | 316 (81.2) | 0.42 |
| tobacco | | | | | | |
| Active tobacco | N (%) | 27 (25.0) | 54 (29.5) | 39 (39.8) | 120 (30.9) | 0.06 |
| Hyperlipidemia | N (%) | 70 (64.8%) | 99 (54.1%) | 44 (44.9%) | 213 (54.8%) | 0.02 |
| Obesity | N (%) | 40 (37.0) | 61 (33.3) | 35 (25.7) | 136 (35.1) | 0.79 |
| CHF on | N (%) | 19 (17.6) | 40 (21.9) | 43 (43.9) | 102 (26.2) | <0.001 |
| presentation | | | | | | |
| MI on | N (%) | 23 (21.3) | 44 (24.0) | 40 (40.8) | 107 (27.5) | 0.003 |
| presentation | | | | | | |
| Chronic renal | N (%) | 1 (0.9) | 4 (2.2) | 13 (13.3) | 18 (4.6) | <0.001 |
| insufficiency | | | | | | |
| ASA use | N (%) | 95 (88.0%) | 160 (87.4%) | 76 (77.6%) | 331 (85.1%) | 0.05 |
| β-blocker use | N (%) | 76 (70.4%) | 121 (66.1%) | 70 (71.4%) | 267 (68.6%) | 0.59 |
| ACE-I use | N (%) | 59 (54.6%) | 112 (61.2%) | 65 (66.3%) | 236 (60.7%) | 0.22 |
| Statin use | N (%) | 66 (61.1%) | 96 (52.5%) | 43 (43.9%) | 205 (52.7%) | 0.04 |
| Fibrate use | N (%) | 3 (2.8%) | 9 (5.0%) | 4 (4.1%) | 16 (4.1%) | 0.67 |
| Prior CABG | N (%) | 8 (7.4) | 18 (9.8) | 9 (9.2) | 35 (9.0) | 0.78 |
| Angiographic | 0 or 1 | 43 (39.8) | 65 (35.5) | 26 (26.5) | 134 (34.5) | 0.12 |
| score | ≧2 | 65 (60.2) | 118 (64.5) | 72 (73.5) | 255 (65.5) | |
| LV systolic | Normal or | 64 (59.3) | 114 (62.3) | 43 (43.9) | 221 (56.8) | 0.02 |
| function | mildly | | | | | |
| | reduced | | | | | |
| | Moderately | 43 (39.8) | 57 (31.1) | 45 (45.9) | 145 (37.3) | |
| | or severely | | | | | |
| | reduced | | | | | |
| Troponin I* | Mean (std) | 6.8 (32.8) | 10.3 (54.2) | 14.4 (47) | 10.4 (47.3) | 0.09 |
| | Median | 0.3 | 0.3 | 0.3 | 0.3 | |
| | 25^{th}-75th | 0.2-1.5 | 0.2-1.0 | 0.2-3.6 | 0.2-1.7 | |
| ESR* | Mean (std) | 19.9 (19.9) | 21.0 (18.7) | 38.5 (32.1) | 25.2 (24.4) | <0.001 |
| | Median | 13 | 14 | 29 | 16 | |
| | 25^{th}-75th | 5-30 | 8-30 | 12-56 | 8-36 | |
| hs-CRP* | Mean (std) | 16.1 (29.9) | 18.6 (29.7) | 40.9 (58.3) | 23.8 (40.5) | <0.001 |
| | Median | 7.4 | 8.7 | 15.0 | 9.2 | |
| | 25^{th}-75th | 3.2-14.1 | 3.8-16 | 5.6-46.5 | 3.8-18.8 | |
| MPO* | Mean (std) | 19.3 (10.4) | 26.0 (21.1) | 29.6 (20.0) | 25.3 (19.1) | <0.001 |
| | Median | 16.0 | 19.3 | 21.8 | 19.3 | |
| | 25^{th}-75th | 12.4-23.6 | 13.7-30.2 | 16.0-39.7 | 13.6-29.1 | |
| PAI-1* | Mean (std) | 33.9 (23.1) | 42.8 (39.2) | 51.9 (37.7) | 42.9 (35.9) | 0.001 |
| | Median | 30.3 | 32.2 | 42.2 | 33.4 | |
| | 25^{th}-75th | 19.0-42.3 | 56.1-17.7 | 23.4-70.4 | 19.0-56.3 | |
| MMP-9* | Mean (std) | 20.7 (20.5) | 26.6 (25.5) | 33.4 (27.3) | 26.9 (25.2) | <0.001 |
| | Median | 14.9 | 18.2 | 23.2 | 18.1 | |
| | 25^{th}-75th | 11.1-22.3 | 12.3-32.8 | 15.4-39.9 | 12.2-31.4 | |

TABLE II
Univariate analysis of baseline characteristics for all-cause
mortality at 24 months in the entire cohort of patients.

| Predictors of death | | |
|---|---|---|
| at 24 months | p-value | Odds ratio and 95% CI |
| Age/10 y | 0.003 | 1.65(1.19, 2.28) |
| Diabetes mellitus | 0.58 | 1.18(0.65, 2.13) |
| Family history of CAD | 0.11 | 0.53(0.24, 1.16) |
| CHF on presentation | <0.001 | 3.64(1.98, 6.68) |
| Aspirin use | 0.55 | 0.79(0.36, 1.72) |
| β-blocker use | 0.34 | 1.38(0.71, 2.71) |
| ACE-i use | 0.37 | 1.33(0.71, 2.48) |
| Statin use | 0.22 | 0.69(0.38, 1.25) |
| Hypertension | 0.92 | 1.04(0.46, 2.34) |
| Obesity | 0.51 | 0.81(0.43, 1.52) |
| Chronic renal insufficiency | 0.003 | 4.66(1.72, 12.64) |
| Hyperlipidemia | 0.34 | 0.71(0.42, 1.35) |
| MI on presentation | 0.17 | 1.54(0.83, 2.88) |
| Active tobacco use | 0.67 | 1.15(0.61, 2.15) |
| Angiographic score | <0.001 | 1.47(1.20, 1.81) |
| LV function | 0.004 | 1.53(1.15, 2.04) |
| TIMP-1 | <0.001 | 7.4(3.5, 15.6) |
| hs-CRP | 0.001 | 1.44(1.16, 1.80) |
| MMP-9 | 0.09 | 0.74(0.52, 1.05) |
| MPO | 0.34 | 1.29(0.77, 2.16) |
| PAI-1 | 0.55 | 0.90(0.65, 1.26) |
| Ratio TIMP-1/MMP-9 | <0.001 | 2.12(1.46, 3.08) |
| ESR | <0.001 | 2.12(1.49, 3.02) |

Abbreviations: ESR = erythrocyte sedimentation rate; hs-CRP = high-sensitivity C-reactive protein; MPO = myeloperoxidase; PAI-1 = plasminogen activator inhibitor-1; MMP-9 = matrix metalloproteinase-9; TIMP-1 = tissue inhibitor of metalloproteinase-1.

TABLE III
Multivariate analysis for all-cause mortality at 24 months
in the entire cohort of patients.*

| Predictors of any death | | |
|---|---|---|
| at 24 months | p-value | Odds ratio and 95% CI |
| Age/10 y | 0.13 | 1.03(0.99, 1.07) |
| CHF on presentation | 0.008 | 2.55(1.28, 5.11) |
| Angiographic score | 0.004 | 1.41(1.12, 1.77) |
| TIMP-1 | 0.001 | 4.93(2.18, 11.16) |

*Logistic regression model involves backward stepwise elimination. Twenty-three clinical and laboratory variables were initially studied (see Table II) by univariate analysis. Only those predictors with p < 0.05 were subsequently entered into a multivariate model, the results of which are displayed above.

TABLE IV
Multivariate analysis for cardiac mortality at 24 months
in the entire cohort of patients*

| Predictors of cardiac death | | |
|---|---|---|
| at 24 months | p-value | Odds Ratio and 95% CI |
| Angiographic score | 0.001 | 1.62(1.23, 2.15) |
| CHF on presentation | 0.006 | 3.11(1.38, 7.03) |
| Ratio TIMP-1/MMP-9 | 0.01 | 1.76(1.15, 2.71) |

*See Table III for methods. Significant (p < 0.05) univariate predictors of cardiac mortality entered into the multivariate model included age, diabetes mellitus, CHF on presentation, chronic renal insufficiency, angiographic score, TIMP-1, MMP-9, ratio TIMP-1/MMP-9, and ESR.

TABLE V
Multivariate analysis for MI at 24 months in the entire
cohort of patients*

| Predictors of MI | | |
|---|---|---|
| at 24 months | p-value | Odds Ratio and 95% CI |
| TIMP-1 | 0.003 | 3.21(1.48, 6.95) |
| LV function | 0.03 | 1.40(1.03, 1.89) |
| Age/10 y | 0.02 | 1.52(1.06, 2.18) |
| Angiographic score | 0.09 | 1.23(0.97, 1.55) |

*See Table III for methods. Significant (p < 0.05) univariate predictors of MI entered into the multivariate model included age, diabetes mellitus, CHF on presentation, beta blocker use on presentation, chronic renal insufficiency, MI on presentation, angiographic score, LV function, TIMP-1, MMP-9, ratio TIMP-1/MMP-9, MPO (p = 0.06) and ESR.

TABLE VI
Multivariate analyses for (A) all-cause mortality, (B) cardiac
mortality and (C) MI at 24 months in the ACS population*
(A)

| Predictors of any death | | |
|---|---|---|
| at 24 months | p-value | Odds ratio and 95% CI |
| Angiographic score | 0.0137 | 1.53(1.09, 2.14) |
| hs-CRP | 0.0143 | 1.53(1.09, 2.16) |
| TIMP-1 | 0.0442 | 2.90(1.03, 8.20) |

(B)

| Predictors of cardiac | | |
|---|---|---|
| at 24 months | p-value | Odds ratio and 95% CI |
| Age/10 y | 0.0329 | 2.31(1.07, 4.99) |
| Ratio TIMP-1/MMP-9 | 0.0164 | 2.05(1.14, 3.69) |

(C)

| Predictors of MI | | |
|---|---|---|
| at 24 months | p-value | Odds ratio and 95% CI |
| Age/10 y | 0.0274 | 1.66(1.06, 2.59) |
| Ratio TIMP-1/MMP-9 | 0.0204 | 1.72(1.09, 2.72) |
| MPO | 0.0068 | 2.73(1.32, 5.64) |
| *See Table III for methods. Significant (p < 0.05) univariate predictors of (A) all-cause mortality (B) cardiac mortality and (C) MI entered into the multivariate model included: (A) age, chronic renal insufficiency, angiographic score, TIMP-1, hs-CRP, MMP-9, ratio TIMP-1/MMP-9, and ESR; (B) age, chronic renal insufficiency (p = 0.06), angiographic score, TIMP-1, hs-CRP, MMP-9, and ratio TIMP-1/MMP-9; (C) age, angiographic score, TIMP-1, hs-CRP (p = 0.06), | | |
| #MPO, and ratio TIMP-1/MMP-9. | | |

TABLE VII
Multivariate analysis for all-cause mortality at 24 months
in the non-ACS chest pain population*

| Predictors of all cause | | |
|---|---|---|
| mortality at 24 months | p-value | Odds ratio and 95% CI |
| Chronic renal insufficiency | 0.0159 | 29.70(1.89, 468.03) |
| Angiographic score | 0.0050 | 2.04(1.24, 3.35) |
| TIMP-1 | 0.0449 | 14.04(1.06, 185.64) |

*See Table III for methods, except that the univariate variable “MI on presentation” was not included in the analysis. Significant (p < 0.05) univariate predictors of all-cause mortality entered into the multivariate model included chronic renal insufficiency, angiographic score, TIMP-1, PAI-1 (p = 0.06), and ESR.

1. A method for characterizing and individual's risk profile of developing vascular disease, the method comprising:
(a) obtaining a biological sample from the individual;
(b) measuring the level of tissue inhibitor of metalloprotein (TIMP) in the biological sample;
(c) comparing the level of TIMP to a predetermined value or a control sample; and
(d) characterizing the individual's risk profile of developing vascular disease based upon the TIMP level of the biological sample in comparison to the predetermined value or control sample.

2. The method of claim 1 wherein the predetermined value is a plurality of predetermined TIMP level ranges and wherein the comparing step comprises determining into which of the predetermined TIMP level ranges the individual falls.

3. The method of claim 2 wherein the TIMP level of the biological sample is elevated compared to the predetermined value or control sample.

4. The method of claim 1 wherein the biological sample is a blood sample, serum sample, plasma sample, tissue sample, or urine sample.

5. The method of claim 1 wherein the vascular disease is artherosclerosis, congestive heart failure, myocardial infarction, chronic renal insufficiency, valvular heart disease or coronary artery disease.

6. The method of claim 1 wherein the tissue inhibitor of metalloprotein (TIMP) is TIMP-1.

7. The method of claim 1 wherein the predetermined value is about 0 ng/ml.

8. The method of claim 1 wherein the predetermined value is about 3 ng/ml or higher.

9. The method of claim 1 wherein the predetermined value is about 5 ng/ml or higher.

10. The method of claim 1 wherein the predetermined value is about 10 ng/ml.

11. The method of claim 1 wherein the vascular disease is artherosclerosis, congestive heart failure, myocardial infarction, chronic renal insufficiency, valvular heart disease or coronary artery disease.

12. A method for characterizing the risk profile for an adverse outcome in a patient diagnosed with vascular disease, the method comprising:
(a) obtaining a biological sample from the patient;
(b) measuring the level of tissue inhibitor of metalloprotein (TIMP) in the sample;
(c) comparing the level of TIMP to a predetermined value or a control sample; and
(d) characterizing the patient's s risk profile of having an adverse outcome based upon the TIMP level of the biological sample in comparison to the predetermined value or control sample.

13. The method of claim 12 wherein the predetermined value is a plurality of predetermined TIMP level ranges and wherein the comparing step comprises determining into which of the predetermined TIMP level ranges the individual falls.

14. The method of claim 13 wherein the TIMP level of the biological sample is elevated compared to the predetermined value or control sample.

15. The method of claim 12 wherein the sample is a blood sample, serum sample, plasma sample, tissue sample, or urine sample.

16. The method of claim 12 wherein the vascular disease is artherosclerosis, congestive heart failure, myocardial infarction, chronic renal insufficiency, valvular heart disease or coronary artery disease.

17. The method of claim 12 wherein the adverse outcome is stroke, myocardial infarction, cardiac death, or non-cardiac death.

18. The method of claim 12 wherein the tissue inhibitor of metalloprotein (TIMP) is TIMP-1.

19. The method of claim 12 wherein the predetermined value is about 10 ng/ml or higher.

20. The method of claim 12 wherein the predetermined value is about 30 ng/ml or higher.

21. The method of claim 12 wherein the predetermined value is about 60 ng/ml.

22. A method for characterizing the risk profile for mortality in an individual, said method comprising
(a) obtaining a biological sample from the individual;
(b) measuring the level of tissue inhibitor of metalloprotein (TIMP) in the biological sample;
(c) comparing the level of TIMP to a predetermined value or a control sample; and
(d) characterizing the individual's risk profile for mortality based upon the TIMP level of the biological sample in comparison to the predetermined value or control sample.

23. The method of claim 22 wherein the predetermined value is a plurality of predetermined TIMP level ranges and wherein the comparing step comprises determining into which of the predetermined TIMP level ranges the individual falls.

24. The method of claim 23 wherein the TIMP level of the biological sample is elevated compared to the predetermined value or control sample.

25. The method of claim 22 or 23 wherein the sample is a blood sample, serum sample, plasma sample, tissue sample, or urine sample.

26. The method of claim 22 wherein the tissue inhibitor of metalloprotein (TIMP) is TIMP-1.

27. The method of claim 22 wherein the predetermined value is about 50 ng/ml or lower.

28. The method of claim 22 wherein the predetermined value is about 80 ng/ml or lower.

29. The method of claim 22 or wherein the predetermined value is 100 ng/ml or lower.

30. A method for guiding therapy in a patient with a vascular disease, said method comprising:
(a) obtaining a baseline level of tissue inhibitor of metalloprotein (TIMP) in a biological sample obtained from the patient,
(b) administering to the patient a treatment for the vascular disease,
(c) obtaining one or more subsequent biological samples from the patient,
(d) measuring TIMP level in the one or more subsequent samples,
(e) comparing the level of TIMP in the one or more subsequent samples with the baseline level; and
(f) determining whether increased dosages or additional or alternative treatments are needed based on TIMP levels obtained in the one or more subsequent samples compared to the TIMP baseline level.

31. The method of claim 30 wherein the absence of a reduction or an insignificant reduction in TIMP levels obtained in the one or more subsequent samples compared with the baseline level, correlates with the need for increased dosage, or additional or alternative treatments.

32. The method of claim 30 wherein the treatment is at least one of anti-coagulant therapy, administration of a lipid lowering drug, anti-platelet therapy, anti-thrombosis therapy, administration of an anti-inflammatory drug, or arterial revascularization.

33. The method of claim 32 wherein the arterial revascularization is surgery or angioplasty.

34. The method of claim 30 wherein the tissue inhibitor metalloprotein (TIMP) is TIMP-1.

35. A kit for identifying an individual at risk for developing vascular disease, for predicting an adverse outcome in a patient diagnosed with vascular disease, for predicting all-cause mortality in an individual, or for guiding therapy in a patient with a vascular disease, the kit comprising an antibody which specifically binds a tissue inhibitor metalloprotein (TIMP), a listing of one or more predetermined values, and instructions for its use.

36. A kit for identifying an individual at risk for developing vascular disease, for predicting an adverse outcome in a patient diagnosed with vascular disease, for predicting all-cause mortality in an individual, or for guiding therapy in a patient with a vascular disease, the kit comprising one or more reagents for detecting cell-bound TIMP by flow cytometry, a listing of one or more predetermined values, and instructions for its use.

37. A kit for identifying an individual at risk for developing vascular disease, for predicting an adverse outcome in a patient diagnosed with vascular disease, for predicting all-cause mortality in an individual, or for guiding therapy in a patient with a vascular disease, the kit comprising one or more probes or primers for detecting TIMP mRNA in a cell sample, a listing of one or more predetermined values, and instructions for its use.

38. A kit for identifying an individual at risk for developing vascular disease, for predicting an adverse outcome in a patient diagnosed with vascular disease, for predicting all-cause mortality in an individual, or for guiding therapy in a patient with a vascular disease, the kit comprising one or more reagents for detecting TIMP in a biological sample by HPLC, a listing of one or more predetermined values, and instructions for its use.

39. The kit of claim 35 wherein the tissue ihibitor metalloprotein (TIMP) is TIMP-1.

40. The kit of claim 35 further comprising one or more containers for collecting or holding a blood, serum, plasma, urine or tissue sample.

41. The kit of claim 35 further comprising a control sample.

42. The method of claim 13 wherein the vascular disease is artherosclerosis, congestive heart failure, myocardial infarction, chronic renal insufficiency, valvular heart disease or coronary artery disease.

43. The method of claim 13 wherein the adverse outcome is stroke, myocardial infarction, cardiac death, or non-cardiac death.

44. The method of claim 13 wherein the tissue inhibitor of metalloprotein (TIMP) is TEMP-1.

45. The method of claim 23 wherein the sample is a blood sample, serum sample, plasma sample, tissue sample, or urine sample.

46. The method of claim 23 wherein the tissue inhibitor of metalloprotein (TIMP) is TIMP-1.

47. The method of claim 23 wherein the predetermined value is 100 ng/ml or lower.

48. The kit of any of claims 36 wherein the tissue inhibitor metalloprotein (TIMP) is TIMP-1.

49. The kit of any of claims 37 wherein the tissue inhibitor metalloprotein (TIMP) is TIMP-1.

50. The kit of any of claims 38 wherein the tissue inhibitor metalloprotein (TIMP) is TIMP-1.
